(19) United States
(12) Patent Application Publication
(10) Pub. No.: US 2009/0149953 A1
(43) Pub. Date: Jun. 11, 2009

(54) FORM STABLE BREAST IMPLANT SIZER AND METHOD OF USE

(76) Inventors: David J. Schuessler, Ventura, CA (US); Erik Torjesen, Santa Babara, CA (US)

Correspondence Address: ALLERGAN, INC., 2525 DUPONT DRIVE, T2-7H, IRVINE, CA, 92612-1599, US

(21) Appl. No.: 12/275,111

(22) Filed: Nov. 20, 2008

Related U.S. Application Data
(60) Provisional application No. 61/012,654, filed on Dec. 10, 2007.

(51) Int. Cl. A61F 2/12

(52) U.S. Cl. 623/8

(57) ABSTRACT

An insertable preformed sizer for a breast implant that regains its form after deformation and insertion into a cavity formed within breast tissue. The sizer is used to evaluate the size of the cavity and help determine proper sizes and shapes of breast implants to use. The implant sizer is intended to be disposable and is made of a cost-efficient material such as a medical grade foam or elastomer. The foam or elastomer material has the ability to be squeezed or collapsed into an extremely small delivery shape and then resiliently expand back to its original shape against the constraining forces of surrounding breast tissue.

### RELATED APPLICATION

This application claims priority to U.S. Provisional Patent Application Ser. No. 61/012,654 filed on Dec. 10, 2007 and which is incorporated herein by reference.

### FIELD OF THE INVENTION

The present invention relates to sizer for a breast implant, and in particular to a breast implant sizer that is both highly compressible and disposable.

### BACKGROUND OF THE INVENTION

Implantable prostheses are commonly used to replace or augment body tissue. In the case of breast cancer, it is sometimes necessary to remove some or all of the mammary gland and surrounding tissue that creates a void that can be filled with an implantable prosthesis. The implant serves to support surrounding tissue and to maintain the appearance of the body. In addition to breast reconstruction surgeries, breast augmentation surgeries involve introducing a soft implant within the breast, sometimes after utilizing a tissue expander or dissector to create or enlarge a void or cavity. In any of these surgeries, the implant is placed within a cavity in the patient's breast.

Soft implants typically include a relatively thin and quite flexible envelope or shell made of vulcanized (cured) silicone elastomer. The shell is filled either with a silicone gel or with a normal saline solution. The filling of the shell takes place before or after the shell is inserted through an incision.

Selecting a particular breast implant with regard to size and shape depends partly on the patient's desires in conjunction with surgeon recommendations. However, the physician must carefully evaluate implant size and contour, incision placement, pocket dissection, and implant placement criteria, with respect to the patient's anatomy and desired physical outcome. One tool that is available for determining the appropriate implant is a temporarily implantable sizer. The sizer is inserted through the actual surgical incision and temporarily placed within the cavity in the patient's breast. The implant sizer enables the physician to actually see the aesthetic effect of implanting a similarly sized and shaped implant, and also helps the physician evaluate the size of the cavity within which the implant will be placed. Such sizers are pre-filled to a constant volume, or may be adjusted in vivo.

One type of implant sizer is adjustably inflated with saline. Once an implant cavity or pocket has been created, the surgeon places an uninflated sizer in one implant pocket and raises the upper half of the O.R. table so that the patient is in an upright position (chest fully upright). The sizer is then inflated gradually to the point that the breasts appear full, but not unnaturally so. In this manner the volume that produces a full but natural breast profile is determined. However, the process is time-consuming and inexact, and is most suitable for saline-filled implants whose volumes can be finely adjusted. These inflatable sizers are not prefilled corresponding to a particular implant, but instead their size and shape is variable.

Another type of implant sizer is constructed in a similar manner as gel-filled implants, with a soft outer silicone shell having a hollow interior filled with a silicone gel. The prefilled nature of these sizers makes their deployment much faster than an adjustable one. Although such a prefilled implant sizer provides the surgeon with an understanding of what a similar implant would look and feel like after implant, there are certain drawbacks. First of all, the gel-filled implant sizers are soft and flexible but relatively incompressible, making them as difficult to pass through a small incision as the actual implant. Secondly, much like placement of a gel-filled implant, its relative lack of form, or squishiness, if you will, may hinder manipulation of the implant sizer into proper orientation and position after insertion within the cavity. Also, the cost of making such implant sizers is relatively high, forcing manufacturers to sell them at a loss. Finally, gel-filled implant sizers are intended to be reusable, and therefore must be carefully sterilized in an autoclave between uses. Not only is this time-consuming, but potentially introduces a source of infection, as well as cross-contamination between patients, if cleaning and sterilization is not done according to manufacturers' recommendations.

Consequently, there remains a need for an implant sizer that overcomes drawbacks with those presently available.

### SUMMARY OF THE INVENTION

The present invention solves many issues with existing insertable breast implant sizers with a preformed sizer that regains its form after deformation and insertion into a cavity formed within breast tissue. The implant sizer is desirably disposable and made of a cost-efficient material such as a medical grade foam or elastomer. The foam or elastomer material has the ability to be squeezed or collapsed into an extremely small delivery shape and then resiliently expand back to its original shape against the constraining forces of surrounding breast tissue.

In one aspect of the invention, a method of evaluating a desired size and shape of implant for a breast implant surgery includes first preparing a patient for a breast implant surgery by forming an incision opening to a cavity within breast tissue. A preformed implant sizer made of a highly compressible material is provided that enables the implant sizer to be compressed from a relaxed size approximating the size of a corresponding implant and having an uncompressed volume, to an insertion size that has an insertion volume less than the uncompressed volume. The surgeon compresses the implant sizer from its relaxed size to its insertion size and inserts it through the incision and into the cavity, permitting it to expand therein. The method includes observing the external characteristics of the breast with the implant sizer inserted therein, and then removing the implant sizer before closing the incision.

The method may involve compressing the implant sizer to an insertion size that has an insertion volume less than about 80%, or even less than about 50%, of the uncompressed volume. The step of compressing comprises folding the implant sizer, and the implant sizer may have at least one hollow on a posterior side thereof that provides a fold relief about which the implant sizer can be folded. Alternatively, the step of compressing comprises rolling the implant sizer into an elongated shape.

An alternative method includes first preparing a patient as described above. An alternative preformed implant sizer is provided made of a collapsible form including an anterior continuous wall and a posterior hollow space that enables the implant sizer to be collapsed from a relaxed size approximating the size of a corresponding implant and having an uncollapsed volume, to an insertion size that has an insertion volume less than the uncollapsed volume. The surgeon collapses the implant sizer from its relaxed size to its insertion size, and inserts it through the incision and into the cavity, permitting it to expand therein, Again, the external characteristics of the breast with the implant sizer are observed before removing it and closing the incision. The collapsible form may be made of highly compressible material, and preferably is at least partly a foam.

Another aspect of the invention is an insertable breast implant sizer comprising a preformed solid form made of a highly compressible material that can be compressed to less than 80% of its uncompressed solid volume, possibly even less than 50% of its uncompressed solid volume. The sizer may be made of a material that is not suitable for long-term implant. In one embodiment, the highly compressible material comprises an inner core with an outer skin, such as a self-skinning foam.

A still further aspect of the invention is an insertable breast implant sizer comprising a preformed collapsible form including an anterior continuous wall and a posterior hollow space. Desirably, the collapsible form is made of highly compressible material, and is at least partly a foam. The collapsible form may include fold reliefs that determine a fold orientation to facilitate collapsing.

The present invention also contemplates a set of insertable breast implant sizers, comprising a marketed collection of at least two differently-sized or shaped preformed collapsible implant sizers. The sizers may be collapsed from a relaxed size approximating the size of a corresponding implant and having a relaxed volume, to an insertion size that has an insertion volume less than the relaxed volume. Each implant sizer in the set is preferably made of a highly compressible material. In one embodiment, the set includes one base portion and a plurality of differently-sized profile portions each which couple to the base portion to form a complete sizer. Unobtrusive handles integrally-formed on each of the two components facilitate junction and separation. Such handles could be used on any of the sizer embodiments described herein to facilitate insertion, orientation, and removal from the pocket.

### BRIEF DESCRIPTION OF THE DRAWINGS

Features and advantages of the present invention will become appreciated as the same become better understood with reference to the specification, claims, and appended drawings wherein:

FIGS. 1A-1C are side, anterior and posterior views, respectively, of an exemplary breast implant sizer of the present invention;

FIGS. 2A-2C are anterior, posterior and vertical section views, respectively, of another exemplary breast implant sizer of the present invention;

FIGS. 3A and 3B are posterior and vertical section views, respectively, of a hollow exemplary breast implant sizer of the present invention;

FIGS. 4A and 4B are posterior and vertical section views, respectively, of an alternative hollow exemplary breast implant sizer of the present invention;

FIGS. 5A and 5B are posterior and vertical section views, respectively, of a hollow exemplary breast implant sizer of the present invention having fold reliefs;

FIG. 6 is a side view of an exemplary two-part breast implant sizer of the present invention;

FIG. 7 is an inferior exploded view of the two-part breast implant sizer of FIG. 6;

FIG. 8 is a perspective view of an exemplary base portion of the two-part breast implant sizer of FIG. 6;

FIG. 9 is a schematic view of a torso of a breast implant patient showing several locations for implant incisions and an implant sizer of the present invention in a relaxed size as well as compressed or rolled to an insertion size to fit through an inframammary incision; and

FIG. 10 is a schematic view of a torso of a breast implant patient shown after insertion of two breast implant sizers of the present invention.

### DETAILED DESCRIPTION OF THE PREFERRED EMBODIMENTS

The present invention provides an improved breast implant sizer that is more easily inserted into a cavity within breast tissue and, because of its relatively low cost, is intended to be disposable after a single use. An insertable sizer is one that is designed to be inserted within breast tissue, i.e., internally, as opposed to an external sizer. The breast implant sizers of the present invention are compressible or collapsible, in contrast with prior implant sizers. The term compressible means that the volume of the sizer can be reduced with the application of external pressure. Prior prefilled breast implant sizers were made of gel-filled sacs which, although they can be distorted, are not compressible. A brief understanding of the technical distinction between compressible and incompressible is appropriate.

In fluid mechanics, an incompressible flow is an idealized solid or fluid flow (isochoric flow) used to simplify analysis. In reality, all materials are compressible to some extent. Note that isochoric refers to flow, not the material property. Indeed, under certain circumstances, a compressible material can undergo (nearly) incompressible flow. All fluids behave incompressibly (to within 5%) when their maximum velocities are below Mach 0.3. A homogeneous, incompressible material is defined as one which has constant density throughout. Thus constant density materials always undergo flow that is incompressible, but the converse is not true.

Technically speaking, water can be compressed, though by only a very little even at high pressures. For practical design purposes, water is considered an incompressible fluid, that is, its density does not change with pressure. The reason anything is compressible is due to how close the atoms are packed together. Air is highly compressible because there is considerable spacing between the atoms, so it is relatively easy to force the atoms closer together. The atoms in liquids are much closer together and considerable pressure is required to make them any closer. Solids also may compress a little under significant pressure.

Therefore, in the context of the present invention, an incompressible material is one which has constant density throughout and exhibits incompressible flow below a velocity of Mach 0.3. All constant density fluids fall within this definition of incompressible materials. Additionally, the volume of incompressible materials cannot be reduced more than a nominal amount (e.g., 5%) when subjected to static compression, or external pressure. Present gel-filled implant sizers are incompressible in this regard, and in order to pass them through a small incision the surgeon must deform one end and essentially extrude the sizer through the incision. Then, once within the breast cavity, the sizer does not automatically rebound to the desired shape but instead must be manipulated into position.

On the other hand, a compressible material in accordance with the present invention is a highly compressible material which does not have constant density throughout and can be statically compressed to reduce its solid volume. Furthermore, highly compressible materials of the present invention desirably can be compressed to reduce their volumes by more than about 5%, at least or less than about 80% of their original solid volumes, and some materials even less than about 50% of their original solid volumes. Finally, in some embodiments, the materials of the present invention are capable of rebounding in vivo into their original preformed shape corresponding to an actual implant.

Exemplary materials of construction for the breast implant sizer of the present invention include biocompatible soft plastics and/or elastomers such as polyvinyl chloride (PVC), thermoplastic elastomers (TPE), and silicone elastomers. An elastomer is a polymer with the property of elasticity. In addition, silicone foams, polyurethane foams, polyethylene foams, and TPE foams are candidates for the highly compressible materials of the breast implant sizers of the present invention. Foams are solids that have trapped gas (air) pockets providing very low density, and are valued for their lightness and compressibility. Foams may be formed from elastomers, but because elastomers are considered solid, not porous materials, they are not foams without a qualifier such as “silicone foam.” The particular physical properties (e.g., compressibility) of any one of these materials can be manipulated depending on the chemical formula and process of formation. It should be understood, therefore, that the present invention encompasses these materials and others which are made to be highly compressible, as defined above.

Desirably, the particular material used is biocompatible and will not subject the patient to an allergic or other type of reaction. However, one of the advantages of the present invention its relatively inexpensive manufacturing cost, permitting the sizers to be disposed of after one use. In this regard, although the exemplary materials are safe for temporary insertion in the body, e.g., they are non-allergenic, they need not be rated for long-term use. In one embodiment of the invention, therefore, the breast implant sizers are made of a material that is not suited for long-term implant purposes. For example, many PVC and polyurethane materials are not cleared (e.g., by the FDA) or well-suited for long-term implant. These materials are typically less expensive which helps to justify their intended ultimate disposal.

It is important to note that in addition to breast implant sizers whose compressibility depends solely on the material properties, the present invention also contemplates collapsible hollow or bowl-like sizers that may or may not be made of a compressible material. Such hollow sizers are typically formed with a continuous wall around the anterior side and one or more hollows or cavities on the posterior side, which hollow enables the sizer to be reduced in size to pass through an incision into the breast cavity, whereupon the sizer resiliently resumes its original shape. For example, certain polymers which are flexible but do not meet the express definition of compressible, as explained above, may be used to form collapsible hollow breast implant sizers of the present invention. Flexible materials that are broadly classed as biocompatible elastomers, as mentioned above, and that are not compressible as defined above, may render a preformed hollow sizer collapsible.

In a general sense, the present invention provides a breast implant sizer made of a preformed compressible or collapsible form. The form may be solid (not hollow), and the material and entire sizer may be compressible, or the form may be hollow, and the material may or may not be compressible, but is at least flexible, rendering the sizer collapsible. In either the entirely compressible or collapsible embodiments, the sizers of the present invention possess the capacity to resiliently expand back to their original forms. Moreover, the sizers have sufficient inherent resiliency to expand after having been inserted into a cavity in breast tissue, or against the confining forces of that tissue.

A particularly useful compressible material for the breast implant sizers of the present invention is termed a self-skinning foam. Such a material forms a less- or non-porous outer layer upon drying or curing, or with the use of a special mold. Forming a breast implant sizer from a self-skinning foam material produces a less- or non-porous outer skin layer surrounding a core of soft porous foam. The concurrent development of the skin and foam core simplifies the manufacturing of the implant sizer by combining what otherwise would be separate steps into one. Moreover, the properties of the outer skin may be designed to facilitate passage through a small incision to the breast, such as by forming a surface that becomes very slippery when wet.

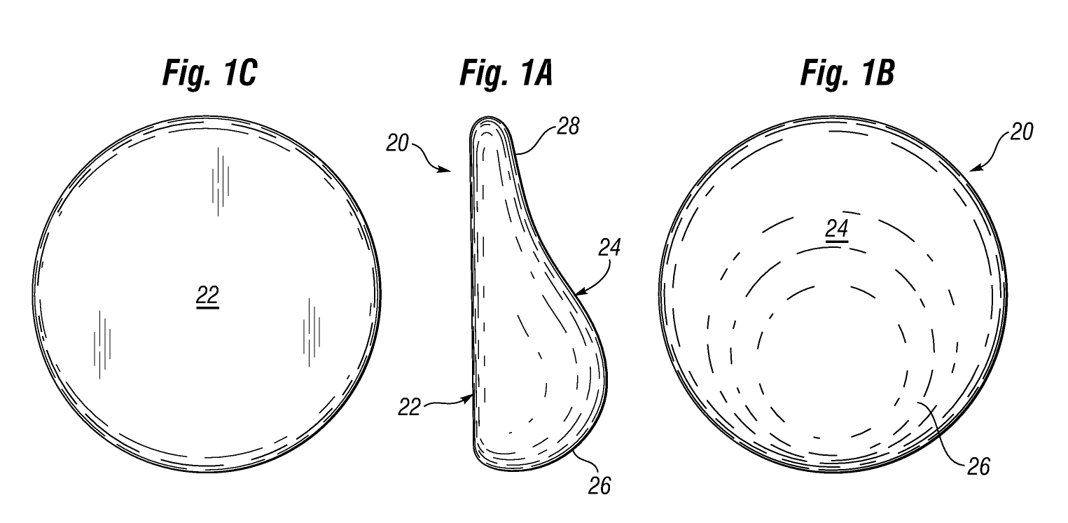

With reference now to FIGS. 1A-1C, a first exemplary breast implant sizer 20 will be described. FIG. 1A is a side view which shows the preferred teardrop-shaped contour of the sizer 20 from the side, with a relatively flat posterior side 22 and a shaped anterior side 24. The anterior side 24 typically includes a somewhat spherical inferior bulge 26 tapering up to a thinner superior edge 28. The posterior 22 and anterior 24 profiles are circular, but may be slightly oval or other shapes as desired. Typically a base dimension is measured across the largest dimension looking at the posterior 22 or anterior 24 profiles. Some sizers have a circular base, as do some implants, though for many the base is oval with a horizontal and vertical dimension.

The shape of the breast implant sizer 20 represents the “classic” breast shape, and is commonly used for the breast implants themselves. In this regard, therefore, the breast implant sizers of the present invention may be shaped in any manner synonymous with the shapes of breast implant, including those that have a round base and a hemispherical profile. Indeed, the sizers of the present invention desirably have shapes corresponding to an actual implant, and the surgeon may wish to try out several contours and/or sizes, to see which provides the most desirable outcome.

The breast implant sizer 20 has a continuous and generally convex, or at least not hollow, exterior surface. Note that a slight concavity or saddle shape is visible in side view on the anterior side 24 just above the inferior bulge 26. This small concave area forms a part of the contour of the anterior side as seen from the side, and is not considered a hollow in terms of certain embodiments described below, primarily because the lateral contour remains convex. Another way to distinguish between a contour that has a concavity in one plane and a “hollow” is to characterize the sizer 20 as having an exterior shape that does not have any indents that would hold water. The sizer 20 is compressible, in that it is primarily formed by a compressible material, as defined above. In one embodiment, the breast implant sizer 20 is formed of a silicone or polyurethane self-skinning foam.

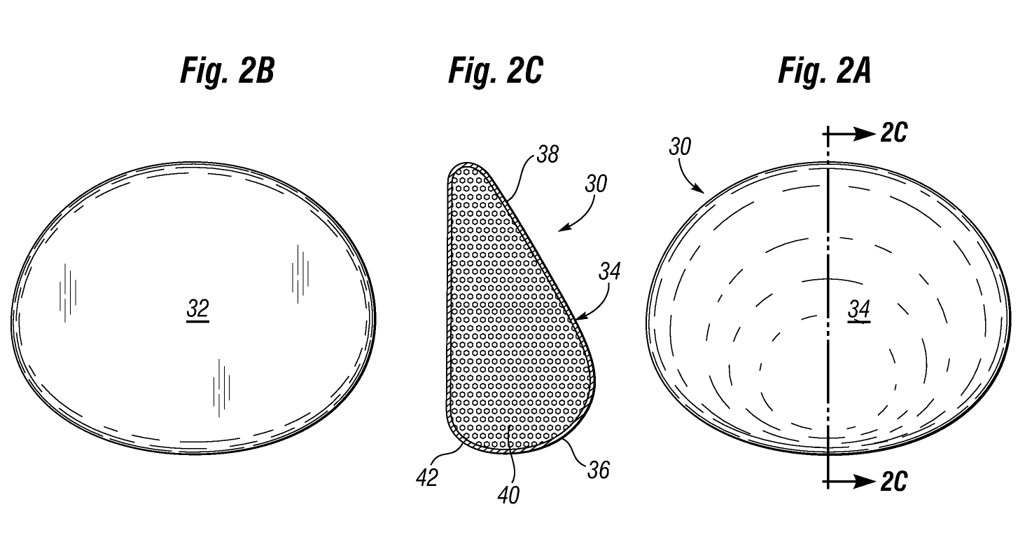

FIGS. 2A-2C are anterior, posterior, and vertical sectional views through an alternative breast implant sizer 30 of the present invention. The sizer 30 again comprises a solid form having a generally flat posterior side 32 and generally convex anterior side 34. Again, the anterior side 34 includes an inferior bulge 36 and a relatively thinner superior portion 38. The exterior shape of the implant sizer 30 is somewhat different than the sizer 20 of FIGS. 1A-1C, in that the vertical-cross-section as seen in FIG. 2C is somewhat more triangular, and less contoured. That is, there is no slight concavity along the superior portion 38 of the anterior side 34. Moreover, as seen in FIGS. 2A and 2B, the front and rear profiles are slightly oval, with the vertical dimension being less than the horizontal dimension.

Additionally, the cross-section of FIG. 2C shows an exemplary construction, with an inner core 40 of porous material and an outer skin 42 of less- or non-porous material. As mentioned above, this construction may be formed by using a self-skinning foam. Alternatively, the outer cover or skin 42 may be separately applied around a preformed core 40. For instance, a foam core molded or cut from block may be inserted into or covered by a shrink-wrap or dip cast outer skin.

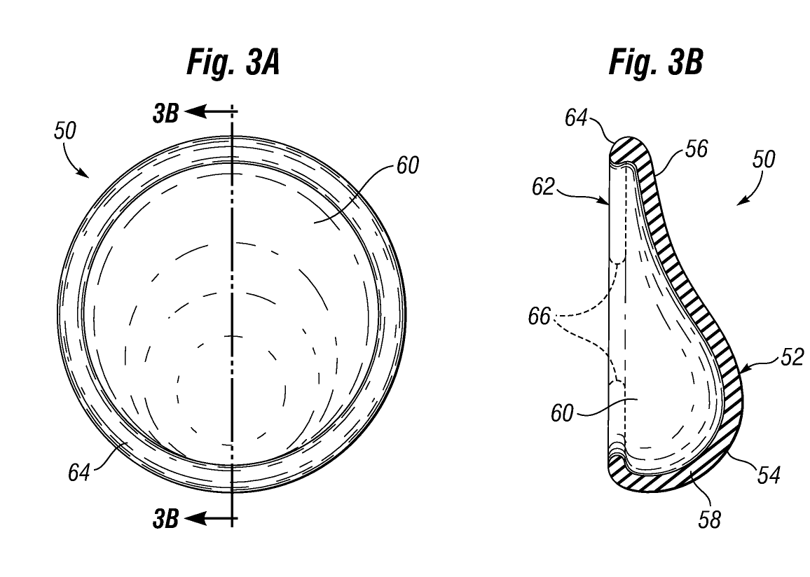

FIGS. 3A and 3B are posterior and sectional views of an exemplary hollow breast implant sizer 50 of the present invention. The overall shape of the sizer 50 is similar to the shape of the sizer 20 in FIGS. 1A-1C, with an anterior side 52 having an inferior bulge 54 and a relatively thinner superior portion 56. Instead of being solid throughout, the sizer 50 comprises a continuous wall 58 having a cavity or hollow 60 defined therein and opening to the posterior side 62. The continuous wall 58 has a substantially constant thickness throughout except for a slightly enlarged peripheral bead or rim 64 that defines the posterior side 62 of the sizer 50. As depicted in FIG. 3B, the peripheral rim 64 is somewhat thicker around its superior aspect than its inferior aspect, though it could be a consistent thickness. Likewise, the rim 64 may be the same thickness as the rest of the wall 58.

The peripheral rim 64 generally defines a planar posterior extent of the sizer 50. In the illustrated version, the rim 64 extends only a slight distance inward so that the opening defined thereby leading to the hollow 60 is maximized. In an alternative shown in phantom at 66, the rim continues farther inward so that the opening is much smaller. Ultimately, the opening need only be sufficiently large to permit passage of air when collapsing and expanding the sizer 50.

As mentioned above, the continuous wall 58 may be made of a compressible material, such as a self-skinning silicone or polyurethane foam. Alternatively, the continuous wall 58 may be a flexible but incompressible material (as defined above) such as biocompatible solids, e.g., silicone elastomers.

The cavity or hollow 60 provided on the posterior side of the flexible implant sizer 50 enables the sizer to be collapsed, rolled or folded into a relatively small size during insertion into the breast cavity. In the general sense, the hollow 60 provides a fold relief, or in other words provides a void into which the outer wall may be collapsed. After insertion, the resiliency of the material of the continuous wall 58 enables the sizer 50 to recover its original shape. Another important characteristic of materials of the present invention is their ability to exert resilient outward pressure on the surrounding breast tissue sufficient to enable the implant sizers to resume their original shape once inserted into the body. The aforementioned rim 64 on the hollow implant sizer 50 functions in this regard to help restore the original profile within the body, especially if it is thickened. This is also in contrast to a prior gel-filled sizer which may require some post-insertion manipulation or molding to form the desired sizer shape, and is certainly not resilient enough to exert outward force on surrounding tissue to assume any particular shape.

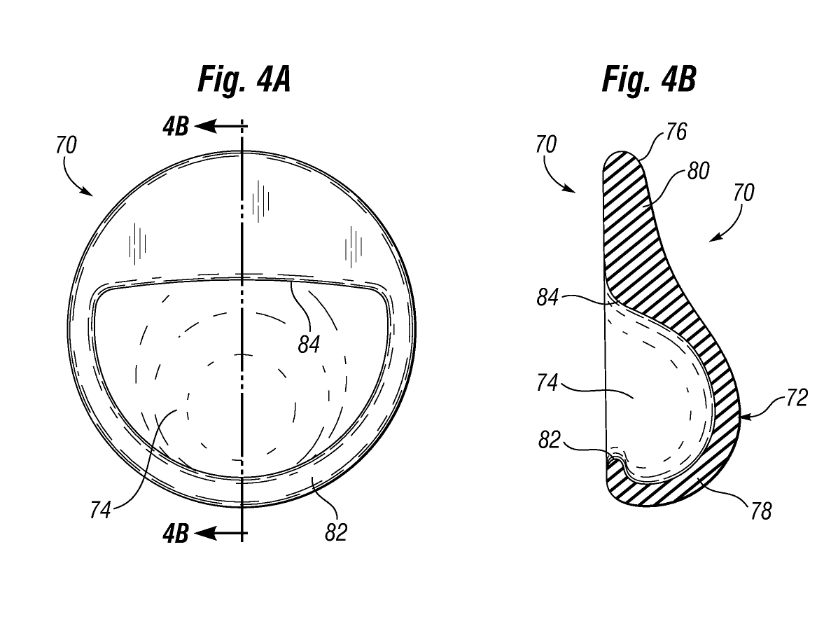

FIGS. 4A and 4B illustrate a still further hollow breast implant sizer 70 of the present invention having a non-uniform wall thickness. The sizer 70 includes a contoured anterior side 72 and the posterior cavity or hollow 74. The hollow 74 is offset in the inferior direction and terminates well below the superior aspect 76 of the implant sizer. A continuous wall 78 includes a portion that surrounds the cavity 74 and a solid superior flange 80.

As seen from the rear in FIG. 4A, the continuous wall 78 includes a partial circular rim 82 along an inferior periphery, and the flange 80 commences at a substantially linear edge 84. The hollow 74 therefore has a generally semi-circular posterior profile. At the time of usage, the sizer 70 may be compressed into a smaller profile by rolling the inferior rim 82 into the cavity 74, and therefore the sizer 70 is partially collapsible.

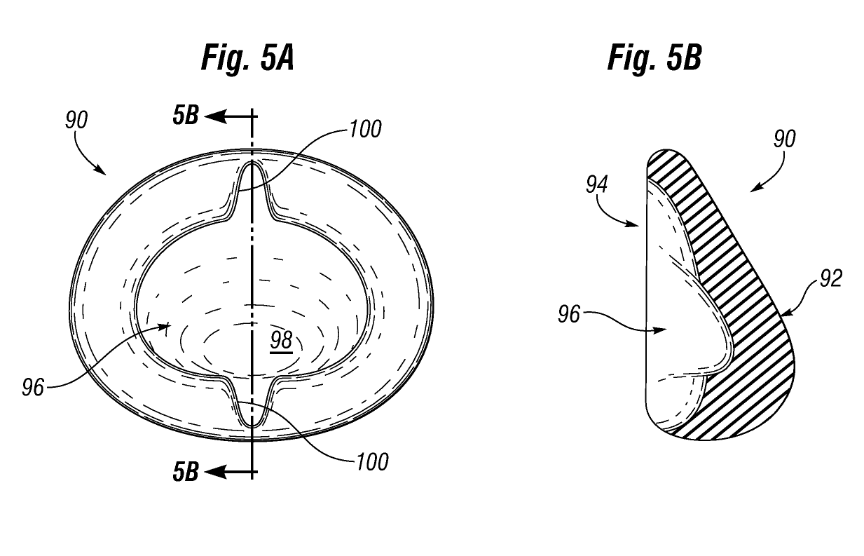

FIGS. 5A and 5B illustrate a still further hollow breast implant sizer 90 of the present invention. As before, the sizer includes a contoured anterior side 92 and a posterior side 94 that includes at least one hollow 96. As seen best from the rear in FIG. 5A, the hollow 96 includes a central relatively deep cavity 98 and a pair of opposed shallow notches 100. The relative depths of the cavity 98 and notches 100 can be seen in FIG. 5B. In the illustrated embodiment, the notches 100 are diametrically opposed across a vertical center line so as to determine a fold orientation, in this case a superior-inferior aligned fold relief. The implant sizer 90 can therefore be folded or rolled up on itself commencing with a rearward fold along the vertical center. Of course, other notches 100 may be provided to facilitate a different compressed or insertion shape, and the illustrated configuration should be seen as exemplary only.

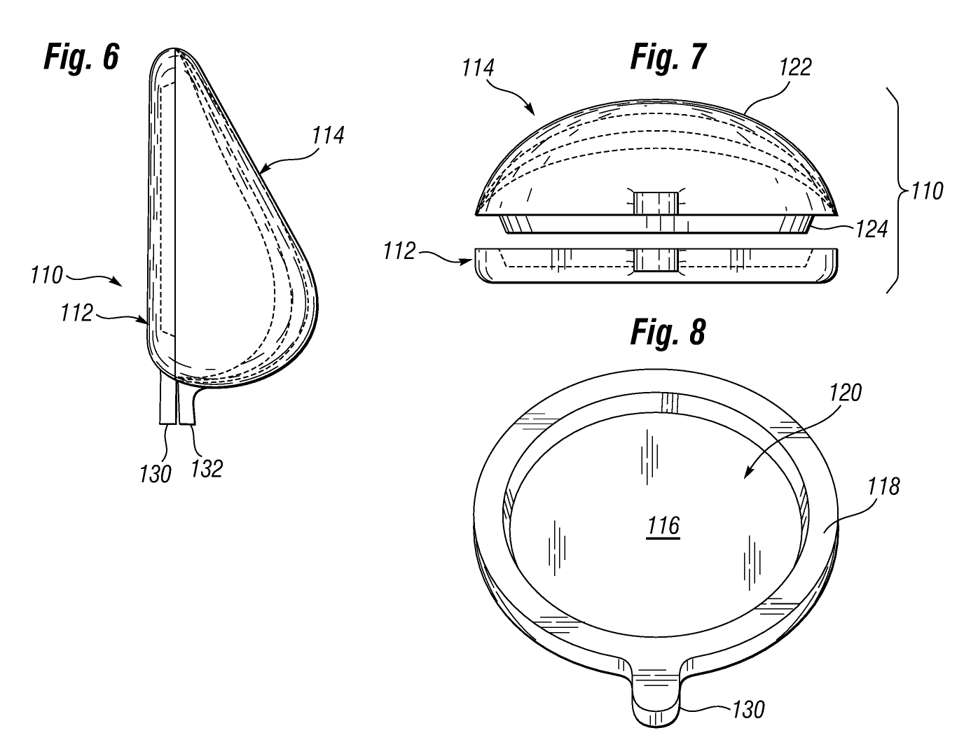

FIGS. 6-8 illustrate an alternative two-part implant sizer having a posterior base portion and an anterior profile portion. With a two-part configuration one base portion can be coupled with a plurality of profile portions, so that only the smaller profile portions need be removed and replaced when evaluating several sizers.

As seen in FIGS. 6-8, an exemplary two-part sizer 110 includes a posterior base portion 112 and an anterior profile portion 114. The base portion 112 defines a plate-like structure with a generally flat posterior wall 116 bordered by an anteriorly-extending flange 118. The flange 118 defines a pocket 120 on the anterior side of the base portion 112 that receives the profile portion 114. In particular, the profile portion 114 presents an anterior preformed surface 122 and a posterior plug or protrusion 124 that fits closely within the pocket 120. A number of different sizes of anterior preformed surfaces 122 can be seen in phantom indicating a variety of different profiles that have the same protrusion 124 and therefore can mate with the base portion 112. The profile portions 114 may be solid and compressible, or hollow and collapsible (and also possibly compressible), in accordance with any of the embodiments described above. Unobtrusive handles 130, 132 integrally-formed on each of the two components facilitate junction and separation. Such handles could be included on any of the sizers of the present invention described elsewhere herein to facilitate insertion, orientation, and removal of the sizer from the pocket.

A set of two-part sizers may include one base portion 112 and a number of profile portions 114 so that the surgeon has the option of evaluating a number of sizers. Initially, the base portion 114 and one of the profile portions 114 are inserted and coupled to form the sizer 110. These components are either compressible or collapsible, as described above, and may be inserted as a unit but more likely separately. The base portion 112 may first be inserted to confirm the size and shape of the pocket dissection. One or more profile portions 114 can then be inserted to confirm the projection and/or volume desired. Once inserted, the base portion 112 remains in place during the evaluation but the first profile portion 114 may be collapsed and removed to be replaced by a second one, and a third, etc. Because the base portion 112 defines the largest dimension, removing and replacing just the profile portions 114 is somewhat easier through the incision.

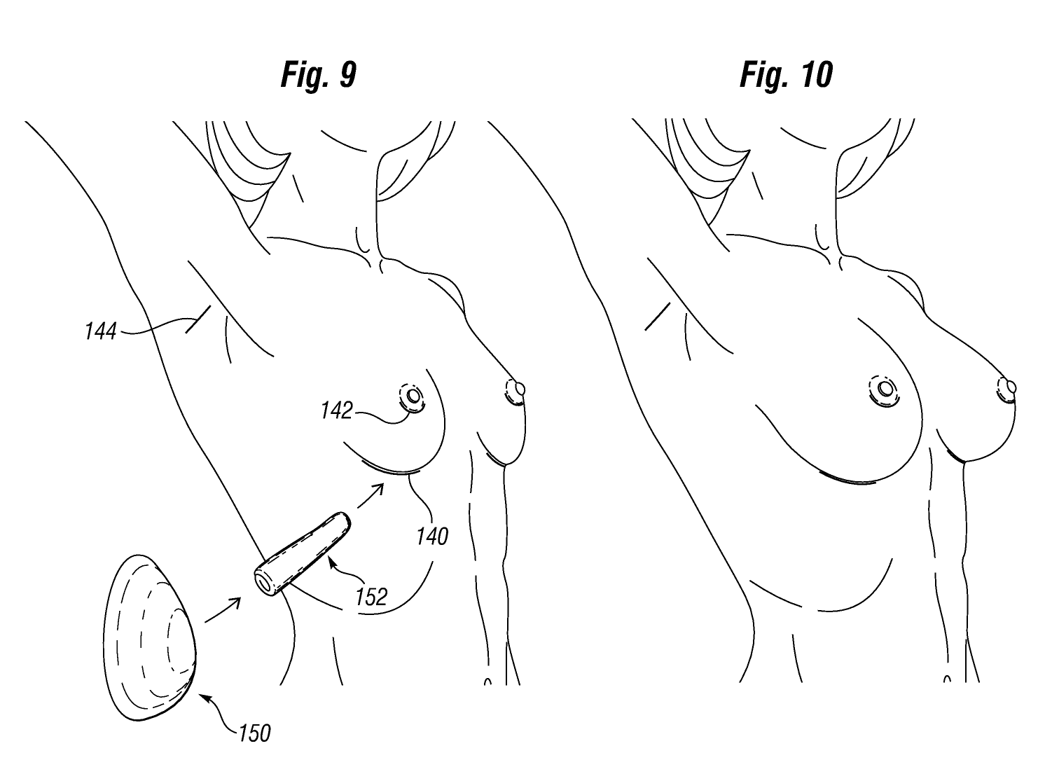

FIGS. 9 and 10 illustrate use of the breast implant sizers of the present invention. In FIG. 9, the torso of a breast implant patient is shown with number of possible incisions used by surgeons. Specifically, the possible incisions include an inframammary incision 140, a periareolar incision 142, and a transaxillary incision 144. It should be understood that the breast implant sizers of the present invention are delivered through the same incision that the eventual implant will be delivered, which is a preference of the surgeon, typically after consultation with the patient. Sizers of the present invention may therefore be inserted through any of the three illustrated incisions 140, 142, 144, or a different incision altogether.

A breast implant sizer 150 is schematically shown in its relaxed size having an uncompressed volume adjacent the torso in FIG. 9. The sizer 150 represents any of the aforementioned sizer configurations, or others which are within the scope of the present invention. Arrows indicate transition from the relaxed size to an elongated insertion size or profile 152 having an insertion volume less than the uncompressed volume. In the illustrated embodiment, the profile 152 is a spirally-rolled cylinder, but may also be simply compressed into an elongated shape, folded, etc. In another embodiment, the sizer 150 collapses with the assistance of a tool, such as a funnel, or upon application of an internal vacuum for configurations such as a hollow form with a small aperture.

The arrows represent delivery of the implant sizer 150 through the inframammary incision 140 of the right breast. The surgeon may insert a compressed sizer 152 into one or both of the breasts, as desired. FIG. 10 illustrates the patient after insertion of a sizer into the cavity in each breast. Advantageously, the sizers have resiliently expanded back to their original forms after insertion and without much if any manipulation by the surgeon. At this point, the surgeon can observe and evaluate whether the size and shape of the selected sizer is appropriate for the patient. Of course, with relatively inexpensive and disposable sizers, a series of sizers can be sequentially inserted if the surgeon is not satisfied at first.

Furthermore, different sizers may be concurrently inserted into each breast to compare the external appearance alongside each other. In this regard, a set of differently-sized breast implant sizers of the present invention may be marketed as a collection for use in preparation for each surgery. Because of the relatively low cost of the sizers, the sets also will be relatively inexpensive, and two of each size and/or profile may be provided.

To illustrate the difficulty of sizing, results of one study using conventional adjustable sizers showed that an average of 189 cc of saline was needed to change one bra cup size, but that the change between cup sizes was not consistent. Increasing an A cup to a C cup required a total of 391 cc, or 196 cc per cup. Moving from a B cup to a D cup required a total of 448 cc, or 224 cc per cup. The largest change, an A cup increasing to a D cup required 437 cc, or 145 cc per cup. The non-linearity of such cup size increases is therefore known, and while the fluid amounts prescribed by various sources help achieve the desired size, they are not exact and differences may occur because of other variables, such as chest wall size, breast tissue and the tissue envelope size. Also, there is a range of implant volumes that would be considered natural for any patient, but while one patient may seek an augmentation that is the small side of natural, another may be interested in something that is larger.

Furthermore, because of their high cost, conventional gel-filled sizers are reused and must undergo the process of sterilization. It would be advantageous to have a set of preformed sizers with a large range of options, without concern for cost. Desirably, the present invention enables a breast implant maker to provide sizers corresponding to every breast implant sold. Sets of sizers corresponding to subsets of implants can therefore be provided at a relatively low cost.

In one embodiment, therefore, breast implant sizers of the present invention are provided in multiple sizes with different profiles, such as the various shapes illustrated herein and others. Most recently, patients choose both an implant size and a profile. Profiles or projections include standard, moderate, mid-range, full, and others. The anterior/posterior shape may be round, or more oval. In conjunction with these choices, the actual volume of the implant ranges greatly. For example, Allergan, Inc. of Irvine, Calif. provides silicone-filled implants in a full projection Style 20 in 23 different implant volumes from 120-800 cc, each with a different base diameter.

The foregoing is just a brief discussion of the variety of different sizes and shapes of implants available. Sets of breast implant sizers of the present invention may be provided corresponding to an entire range of implant styles, such as Style 20 from Allergan, Inc. and others, or may be provided in a sampling of different sizes within one particular style. Alternatively, several similar sizes across different styles may be provided in one set so that the surgeon can first make an estimate of the approximate size, and then try out a number of different styles. Furthermore, custom sets of sizers may be ordered by a surgeon depending on an initial consultation with a particular patient. As the reader will understand, numerous permutations of these sets are possible and contemplated, and an exhaustive list is not necessary.

Although the invention has been described and illustrated with a certain degree of particularity, it is understood that the present disclosure has been made only by way of example, and that numerous changes in the combination and arrangement of parts can be resorted to by those skilled in the art without departing from the scope of the invention, as hereinafter claimed.

What is claimed is:

1. A method of evaluating a desired size and shape of implant for a breast implant surgery, comprising:
preparing a patient for a breast implant surgery, including forming an incision opening to a cavity within breast tissue;
providing a preformed implant sizer made of a highly compressible material that enables the implant sizer to be compressed from a relaxed size approximating the size of a corresponding implant and having an uncompressed volume, to an insertion size that has an insertion volume less than the uncompressed volume;
compressing the implant sizer from its relaxed size to its insertion size;
inserting the compressed implant sizer through the incision and into the cavity, and permitting it to expand therein;
observing the external characteristics of the breast with the implant sizer inserted therein; and
removing the implant sizer from the cavity.

2. The method of claim 1, including compressing the implant sizer to an insertion size that has an insertion volume less than about 80% of the uncompressed volume.

3. The method of claim 2, including compressing the implant sizer to an insertion size that has an insertion volume less than about 50% of the uncompressed volume.

4. The method of claim 1, wherein the step of compressing comprises an act selected from the group consisting of:
folding the implant sizer, and
rolling the implant sizer into an elongated shape.

5. The method of claim 1, wherein the highly compressible material is made of a self-skinning foam.

6. A method of evaluating a desired size and shape of implant for a breast implant surgery, comprising:
preparing a patient for a breast implant surgery, including forming an incision opening to a cavity within breast tissue;
providing a preformed implant sizer made of a collapsible form including an anterior continuous wall and a posterior hollow space that enables the implant sizer to be collapsed from a relaxed size approximating the size of a corresponding implant and having an uncollapsed volume, to an insertion size that has an insertion volume less than the uncollapsed volume;
collapsing the implant sizer from its relaxed size to its insertion size;
inserting the collapsed implant sizer through the incision and into the cavity, and permitting it to expand therein;
observing the external characteristics of the breast with the implant sizer inserted therein; and
removing the implant sizer from the cavity.

7. The method of claim 6, wherein the implant sizer includes at least one hollow on a posterior side thereof that provides a fold relief about which the implant sizer can be folded.

8. The method of claim 6, wherein the step of collapsing comprises one of rolling the implant sizer into an elongated shape and folding the implant sizer.

9. The method of claim 6, wherein the collapsible form is made of highly compressible material.

10. The method of claim 9, wherein the highly compressible material is at least partially a foam.

11. An insertable sizer for a breast implant, comprising:
a preformed solid form made of a highly compressible material that can be compressed to less than about 80% of its uncompressed solid volume.

12. The sizer of claim 13, wherein the highly compressible material can be compressed to less than about 50% of its uncompressed solid volume.

13. The sizer of claim 9, wherein the highly compressible material is a self-skinning foam.

14. An insertable sizer for a breast implant, comprising:
a preformed collapsible form including an anterior continuous wall and a posterior hollow space.

15. The sizer of claim 14, wherein the collapsible form is made of highly compressible material.

16. The sizer of claim 15, wherein the highly compressible material is at least partially a foam.

17. The sizer of claim 14, wherein the collapsible form includes fold reliefs that determine a fold orientation.

18. A set of insertable breast implant sizers, comprising:
a marketed collection of at least two differently-sized or shaped preformed collapsible implant sizers that may be collapsed from a relaxed size approximating the size of a corresponding implant and having a relaxed volume, to an insertion size that has an insertion volume less than the relaxed volume.

19. The set of claim 18, wherein each implant sizer in the set is made of a highly compressible material.

20. The set of claim 18, wherein the set includes one base portion and a plurality of differently-sized profile portions each which couple to the base portion to form a complete sizer.
